(19) United States
(12) Patent Application Publication
(10) Pub. No.: US 2009/0050139 A1
(43) Pub. Date: Feb. 26, 2009

(54) LIQUID MEDICINE EJECTION DEVICE

(75) Inventors: Shinji Watanabe, Kawasaki-shi (JP); Mitsuru Imai, Chichibu-shi (JP)

Correspondence Address: FITZPATRICK CELLA HARPER & SCINTO, 30 ROCKEFELLER PLAZA, NEW YORK, NY, 10112, US

(73) Assignee: CANON KABUSHIKI KAISHA, Tokyo (JP)

(21) Appl. No.: 11/908,566

(22) PCT Filed: Mar. 28, 2006
(86) PCT No.: PCT/JP2006/307018

§ 371 Date: Sep. 13, 2007

(30) Foreign Application Priority Data
Mar. 30, 2005 (JP) 2005-096590

(51) Int. Cl. A61M 11/00

(52) U.S. Cl. 128/200.14; 128/202.13

(57) ABSTRACT

A liquid medicine ejection device for ejecting a liquid medicine as liquid droplets to be inhaled by a user includes means for providing information on the ejection to the user without providing means for determining a state of the ejection when a preliminary ejection operation is carried out, the preliminary ejection operation ejecting an amount of the liquid medicine smaller than an amount ordinarily ejected. The liquid medicine ejection device enables improved reliability of ejection and comfort of a user by carrying out preliminary ejection before inhalation and by making it possible for the user to visually or auditorily check an ejection state of minute liquid droplets with a relatively simple structure to see whether or not the preliminary ejection is carried out correctly.

### TECHNICAL FIELD

The present invention relates to a liquid medicine ejection device such as a medicine ejection device structured so as to be carried by a user, and is used for ejecting a medicine as minute liquid droplets to be inhaled by the user. More particularly, the present invention relates to a technology for safer use of a liquid medicine ejection device for healthcare or the like.

### BACKGROUND ART

In recent years, medical and scientific advances increase average life span, which results in the advent of the aging society. On the other hand, eating habits and living environments change, environmental pollution increases, and new illnesses and infectious diseases due to viruses and bacteria are discovered, raising concerns about health. Particularly, in developed countries, there is a problem in that the number of patients of lifestyle-related diseases such as diabetes and high blood pressure is increasing.

On the other hand, the number of medical institutions has not increased enough to accommodate such the increased patients, and in some areas there is no medical institution to go to. This has raised concerns about how to handle these problems in the future involving policies.

To be specific, among diabetics whose number is increasing recently, patients of insulin-dependent diabetes called Type I must administer insulin regularly since their pancreata do not secrete insulin. Insulin is now administered by hypodermic injection, which is burdensome to users both physically and mentally.

In order to reduce such burdens of the users, a pen-type injector has been developed, which has a thin needle and thus the users are insensible to pain. However, since it is often the case that Type I diabetics lead ordinary lives except that insulin must be administered to them regularly, even if the injector is a pen-type, there is a reluctance to give themselves an injection in company, and thus, it is difficult to administer insulin at appropriate times.

As a result, there is a fear in that, in such a method, users are not appropriately treated. However, a medicine ejection device with which a user inhales a medicine realizes treatment of the user while making use of an information database such as electronic charts. Such a medicine ejection device has memory means for storing information on each individual user including the user's charts and prescriptions. The medicine ejection device is also a portable terminal provided with an inhaler with which a user inhales a medicine ejected as minute liquid droplets, and has ejection control means for controlling the inhaler according to an inhalation profile to eject a medicine such that a user can inhale the medicine according to information in the prescriptions.

Such a medicine ejection device enables accurate control of a dose of the medicine and intervals of administration in accordance with a prescription, and enables appropriate control of the ejection according to an inhalation profile of each individual user to administer the medicine with efficiency. With such the medicine ejection device, since, unlike a conventional case, medical instruments such as an injector are not necessary when a medicine is administered, the device can be operated easily without expert knowledge, and pain of the user caused by an injection needle can be removed.

Generally, deposition of minute liquid droplets of a medicine in the lung depends on the diameter of the droplets. In particular, delivery of a medicine to alveoli pulmonis, which are in the deep lung requires liquid droplets having diameters in the range of 1 to 5 μm with a narrow size distribution, and a device which can administer such the droplets with high repeatability is under development.

On the other hand, since such a device is used for administering a medicine to a human body, more appropriate handling of a malfunction of a medicine ejection unit is required. Even if the medicine ejection unit in the medicine ejection device is set in place, when the medicine ejection device itself is required to be carried, there is a fear in that unsatisfactory ejection is caused due to an exhausted battery or the like. There is also such a risk that a necessary amount of a medicine cannot be inhaled due to an insufficient amount of the remaining medicine. In this way, unlike administration using an injector, since an inpicked up state of a medicine cannot be checked, there is a fear in that a user is anxious about the reliability of the ejection.

In order to solve such a problem without fail, a method has been proposed, where the reliability of such a device is improved by carrying out preliminary ejection and by detecting ejection of a medicine by detecting means such as a semiconductor sensor (see Japanese Patent Application Laid-Open No. 2004-97617).

Further, in the field of printing technology, there is a technique where an acoustic wave detecting device is used as means for checking whether a nozzle is in a satisfactory state or not (see Japanese Patent Application Laid-Open No. 2004-167773).

### DISCLOSURE OF THE INVENTION

Example of the above-mentioned detecting means for detecting ejection of a medicine includes optical means for detecting reflected light, refracted light, transmitted light, or scattered light due to an ejected medicine atmosphere, by using natural light or a laser beam. Alternately, there are employed a method of detecting temperature changes due to ejection of a medicine by using infrared radiation, and a method of using a humidity sensor for detecting capacitance changes or impedance changes in a medicine atmosphere.

However, in those methods, output of various kinds of sensors is processed by determining means such as calculating means to determine the ejection state. Based on results of the determination, the next processing is carried out or announcement to the user is made. For example, when the result of the decision is “no problem,” the next processing is carried out without a warning or a display only displays that there is no problem.

However, there remains a fear in that determination using such a sensor and determining means may be a misjudge due to the environment, aging of the sensor, or the like. However, the user has no choice but to trust the result of the determination of the device, and, after all, the user's feelings of anxiety cannot be eliminated.

In view of the above-mentioned problems, according to the present invention, a liquid medicine ejection device such as a medicine ejection device is characterized by including means for providing information on the ejection to the user without providing means for determining a state of the ejection when a preliminary ejection operation is carried out, the preliminary ejection operation ejecting an amount of the liquid medicine smaller than an amount ordinarily ejected.

According to an aspect of the present invention, the means for providing information on the ejection to the user without providing means for determining a state of the ejection includes means for enabling the ejection to be visually checked from outside.

Further, according to another aspect of the present invention, the means for providing information on the ejection to the user without providing means for determining a state of the ejection further includes means for collecting sound generated upon ejection; and means for outputting the collected sound to the user.

In any of the above-mentioned structure, the liquid medicine ejection device, further includes a passage means serving also as a flow path of airflow containing liquid droplets when the liquid droplets are inhaled. The liquid medicine ejection device of the present invention is typically structured so as to be carried by a user.

According to the present invention, preliminary ejection is carried out before inhalation, and a user himself/herself can sensorily check the ejection state of the liquid droplets with a relatively simple structure to see whether or not the preliminary ejection is carried out correctly, and thus, the reliability of the ejection is improved and comfort can be given to the user.

Other features and advantages of the present invention will be apparent from the following description picked upn in conjunction with the accompanying drawings, in which like reference characters designate the same or similar parts throughout the figures thereof.

### BRIEF DESCRIPTION OF THE DRAWINGS

FIG. 1 is a front view showing a medicine ejection device according to an embodiment of the present invention in such a state that a medicine ejection unit and a mouthpiece are not attached thereto.

FIG. 2 is a perspective view illustrating a medicine ejection device according to a first embodiment of the present invention.

FIG. 3 is comprised of FIGS. 3A and 3B showing flow charts of an operation of a medicine ejection device according to a second embodiment of the present invention.

FIG. 4 is a front view showing a medicine ejection device according to a third embodiment of the present invention in such a state that a medicine ejection unit and a mouthpiece are not attached thereto.

FIG. 5 is comprised of FIGS. 5A and 5B showing flow charts of an operation of a medicine ejection device according to a fourth embodiment of the present invention.

FIG. 6 is a front view showing a medicine ejection device according to a fifth embodiment of the present invention in such a state that a medicine ejection unit and a mouthpiece are not attached thereto.

FIG. 7 is comprised of FIGS. 7A and 7B showing flow charts of an operation of a medicine ejection device according to a sixth embodiment of the present invention.

### BEST MODE FOR CARRYING OUT THE INVENTION

Preferred embodiments of the present invention will now be described in detail in accordance with the accompanying drawings.

The present invention provides a liquid medicine ejection device for ejecting a liquid medicine as liquid droplets to be inhaled by a user, including: means for providing information on the ejection to the user without providing means for determining a state of the ejection when a preliminary ejection operation is carried out, the preliminary ejection operation ejecting an amount of the liquid medicine smaller than an amount ordinarily ejected.

In the present invention, information on ejection is information which can be used to see if no abnormality is caused in an ejecting operation of the liquid ejection device. More specifically, such the information includes a state of an acoustic wave generated by an ejection portion in an ejecting operation and a state of a mist generated as a result of liquid ejection.

The means for providing information in the present invention enables a user to check such a state. The means is structured such that a user can directly or indirectly check the ejecting operation sensorily (in particular visually or auditorily).

It is to be noted that, however, in the means for providing information, a structure for providing results of determination by the determining means in the liquid medicine discharging device is not included in the main invention, because the structure does not provide comfort of the user.

To be specific, structures of the present invention are disclosed below.

According to an aspect of the present invention, the means for providing information on the ejection to the user without providing means for determining a state of the ejection includes means for enabling the ejection to be visually checked from outside.

According to another aspect of the present invention, the means for providing information on the ejection to the user without providing means for determining a state of the ejection includes means for taking an image in the vicinity of an ejection portion; and means for displaying the picked up image.

Further, according to another aspect of the present invention, the means for providing information on the ejection to the user without providing means for determining a state of the ejection includes means for collecting sound generated upon ejection; and means for outputting the collected sound to the user.

For a full understanding of embodiment modes of the present invention, specific embodiments of the present invention are now described in detail with reference to the figures.

### Embodiment 1

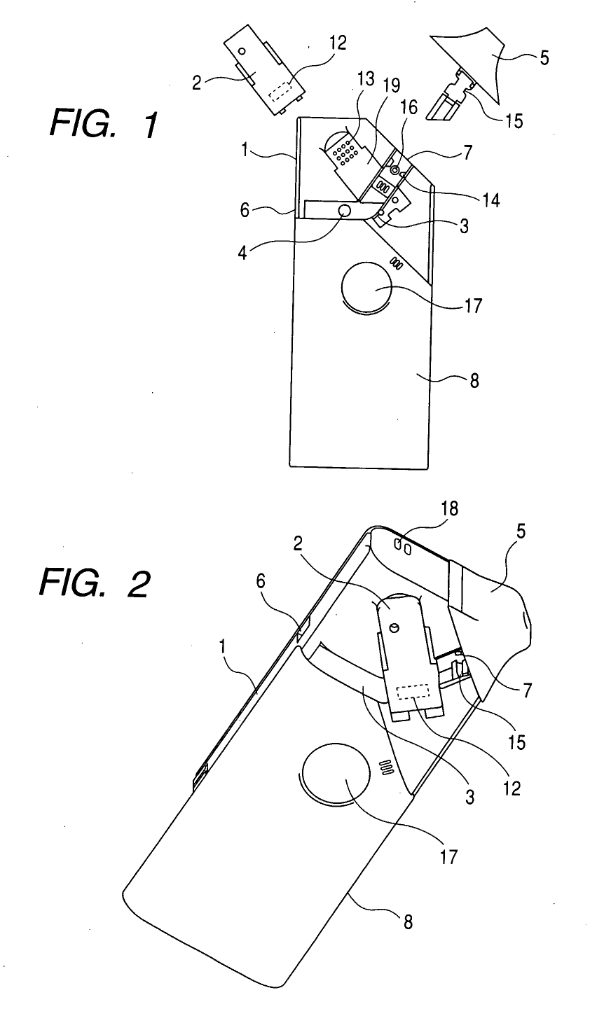

FIGS. 1 and 2 illustrate a structure of a main portion of a first embodiment of the present invention. In the figures, reference numeral 1 denotes a medicine ejection device body and reference numeral 2 denotes a medicine ejection unit including a reservoir for containing a medicine. The medicine ejection unit 2 has a head portion (ejection portion, not shown) for ejecting the medicine on the side over the plane of the drawing. The medicine ejection unit 2 can be detachably attached to an ejection unit attaching portion 19 provided on the side of the device body 1. When the medicine ejection unit 2 is attached to the ejection unit attaching portion 19, an electrically connecting portion of the medicine ejection unit 2 is connected to an electric contact 13 of the ejection unit attaching portion 19 to receive electric power or various kinds of electric control signals from a battery or a control circuit on the side of the device body 1.

Reference numeral 3 denotes an air flow path provided in the medicine ejection device 1. Although, in the figures, the air flow path 3 is shown bent taking in consideration the arrangement of other components, it may be formed straight. When the medicine ejection unit 2 is attached to the ejection unit attaching portion 19, the head portion 12 is exposed to the air flow path 3. Reference numeral 4 denotes light emitting means such as a high intensity LED which is provided on the air flow path 3, for emitting a light beam across the air flow path 3 in an appropriate direction. The light emitting means 4 is disposed in the vicinity of the head portion 12 in order to illuminate an atmosphere of mist-like liquid droplets ejected from the head portion 12. Reference numeral 5 denotes a mouthpiece used when a user inhales the medicine. The mouthpiece 5 can also be detachably attached to the device body 1 with a recession 15 thereof being in engagement with a protrusion 14 provided in the air flow path 3. Since it is desirable to make the medicine ejection unit 2 and the mouthpiece 5 disposable or periodically replaced from a hygienic viewpoint, the medicine ejection unit 2 and the mouthpiece 5 may be structured to be integral with each other.

Reference numeral 6 denotes an inlet (air intake opening) while reference numeral 7 denotes an outlet (air discharge opening) of the air flow path 3. When the mouthpiece 5 is attached to the outlet 7 and a power on/off button 17 is pressed to start the inhalation operation, the medicine is ejected from the head portion 12 of the medicine ejection unit 2 and liquid droplets of the medicine goes from the inlet 6 through the air flow path 3 toward the outlet 7. Inhalation causes air to flow from the inlet 6, and the generated air flow makes the liquid droplets of the medicine picked up in a human body through the mouthpiece 5 attached to the outlet 7. Here, inhalation by the user starts. When negative pressure (related to the inhalation speed or flow rate) sensed by a negative pressure sensor 16 reaches a region where the medicine can be ejected, medicine ejection from the head portion 12 starts under the control of the control circuit.

It is to be noted that the head portion is preferably structured so as to be able to eject liquid droplets having liquid droplet diameters ranging between 1 to 5 μm and having a narrow flow rate distribution. In particular, an inkjet head capable of more accurate droplet control is preferable, and in particular, a bubble jet head which enables nozzles to be high density is preferably used to eject a large amount of minute liquid droplets.

Further, reference numeral 8 denotes a sliding cover 8. By sliding and closing the cover 8, an open portion over the air flow path 3 is closed and the medicine ejection unit 2 is fixed. FIGS. 1 and 2 illustrate an open state. A power on/off switch may be actuated in association with the closing of the cover 8.

In this embodiment, normal ejection when the medicine is inhaled is started by detecting means such as the negative pressure sensor 16 in association with the inhalation operation. On the other hand, when preliminary ejection for checking the ejection state of the head portion 12 is carried out, the ejection is started by, for example, operating a switch by which the user can arbitrarily instruct the start. The amount of the preliminary ejection may be set on the side of the device to be equal to or lower than the amount of the normal ejection, or may be adjusted by the user's operation of the switch. Here, the light emitting means 4 may emit light simultaneously with the start of the preliminary ejection, or the user may make the light emitting means 4 to emit light by operating another switch.

Further, light detecting means may be provided to control the amount of emitted light according to the peripheral brightness.

In the preliminary ejection, unlike the case of the normal ejection, air flow is not generated and the ejected medicine drifts like a mist in the air flow path 3 near the head portion 12. The medicine liquid droplets ejected at the time of the preliminary ejection and drifting like a mist in a relatively dark place is illuminated by the light emitting means 4 and the state is observed from the inlet 6. As a result, the user can visually check the state to see whether or not the preliminary ejection is carried out. Since both the inlet 6 and the outlet 7 of the air flow path 3 which also serves as passage means are open to the outside of the medicine ejection device 1, the inside of the air flow path 3 can be visually observed through them. However, since, normally, the mouthpiece 5 is connected to the outlet 7, it is more convenient to carry out the observation from the side of the inlet 6. Here, when it can be confirmed that the preliminary ejection is carried out correctly, the operation may proceed to the normal ejection to carry out the inhalation of the liquid medicine. When it is not confirmed that the preliminary ejection is carried out correctly, appropriate measures may be taken such as check of the preliminary ejection once again. When a successful preliminary ejection still cannot be confirmed, the medicine ejection unit 2 may be replaced.

According to this embodiment, the user can visually check the ejection state of the minute liquid droplets with a relatively simple structure where the medicine ejection unit 2 is controllable so that the preliminary ejection can be carried out and the light emitting means 4 is provided so as to check the ejection state. Therefore, the reliability of the normal ejection is further improved and comfort can be given to the user.

### Embodiment 2

Next, a second embodiment of the present invention is described. An outer structure of this embodiment is also as illustrated in FIGS. 1 and 2. In this embodiment, control means is structured so that the preliminary ejection and the normal ejection are carried out unlike those in the first embodiment.

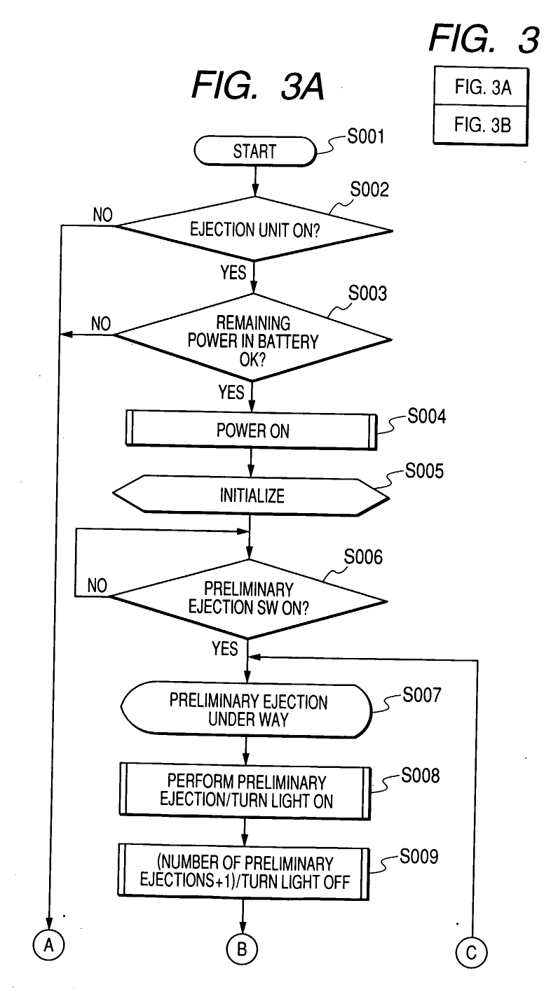

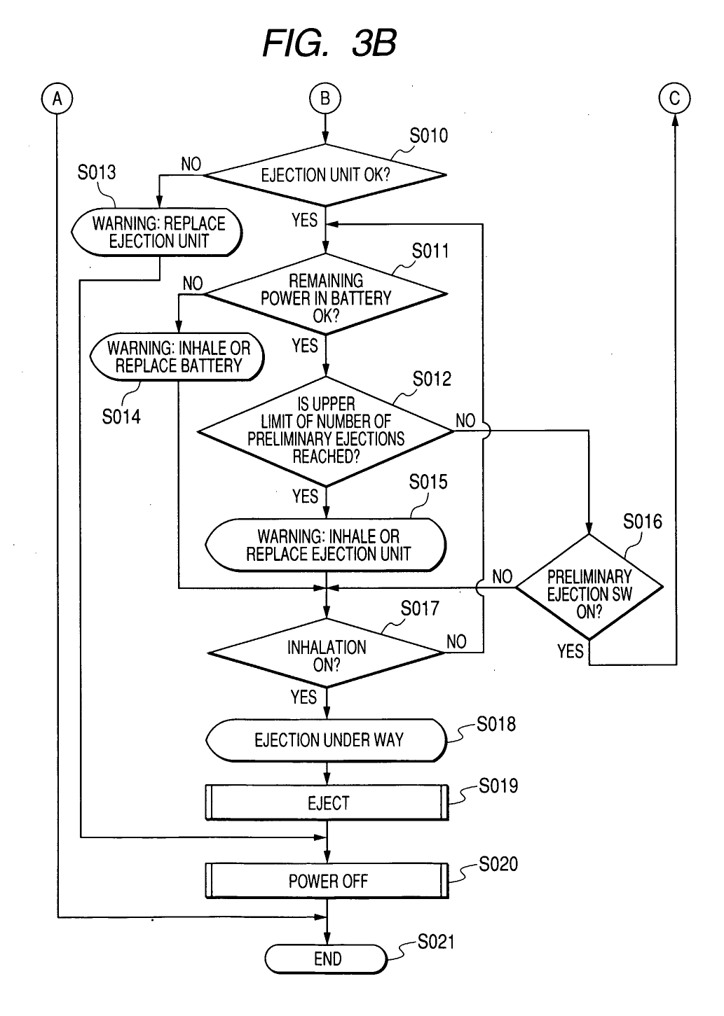

FIGS. 3A and 3B are operation flow charts of a medicine ejection device 1 of this embodiment. First, the device 1 enters a use starting state by operation such as pressing a power on/off switch by a user (Step 001). After the use of the device starts, the device 1 checks whether or not there is a medicine ejection unit 2 (Step S002). If the answer is NO, the process proceeds to an end as it is (Step S021). Detecting means for detecting whether or not there is the medicine ejection unit 2 at an ejection unit attaching portion 19 can be materialized by, for example, when the medicine ejection unit 2 ejects the medicine by a bubble jet system, measuring the resistance of a heater as ejection energy generating means.

When there is the medicine ejection unit 2, the remaining power in the battery is checked (Step S003). If the answer is NO, the process proceeds to the end (Step 021). In this case, display means 18 (see FIG. 2) or the like may be used to provide an indication urging the user to replace or recharge the battery. When it is judged that the remaining power in the battery is enough for at least one preliminary ejection and one inhalation operation, the power is turned on (Step S004) and the device is initialized (Step S005). When the medicine ejection unit 2 is of a one-time-use disposable type, the number of the preliminary ejection operations is reset in the initialization at Step S005.

After the reset at Step S005 is completed, in order to improve the reliability of the ejection, it is necessary to carry out the preliminary ejection operation at least once. Therefore, judgement whether a switch for the preliminary ejection is turned on or not at Step S006 may be automated.

When start of the preliminary ejection is instructed automatically or manually, display is carried out to notify the user that the preliminary ejection is under way (Step S007), and, in order to start the check operation, the preliminary ejection from the ejection head 12 and emission of light by the light emitting means 4 are carried out (Step S008). Here, the user visually checks the state of the preliminary ejection through the flow path 3.

In the preliminary ejection, it is assumed that the conditions of the ejection such as drive frequency and drive time suitable for visual observation differ depending on the kind of the medicine, the prescription, an additive, and the like. If the conditions of the ejection change, the amount of the medicine and the electric power consumed in the preliminary ejection also change. In order to reserve an amount of the medicine necessary for at least one inhalation, a controlling function operated by the user to limit the number of the preliminary ejections is necessary. It is desirable that the amount of the medicine consumed in the preliminary ejection is smaller than that necessary for the inhalation. The preliminary ejection is allowed to be carried out up to (C−A)/B (>1) times, wherein A is an amount necessary for the inhalation, B is an amount consumed in the preliminary ejection, and C is the total amount of the medicine contained in the medicine ejection unit 2 or the maximum amount used in one ejection. After the preliminary ejection ends, the number of the preliminary ejection operations is counted and the light emitting means 4 is shut off to end the check operation (Step S009).

Since there is a fear in that the preliminary ejection causes something unusual in the medicine ejection unit 2 to pose a problem in inhaling, checking means for the medicine ejection unit 2 is provided in the medicine ejection device 1 to check the medicine ejection unit 2 after the preliminary ejection ends (Step S010). The checking means itself may be the same as the means for checking whether or not there is the medicine ejection unit 2 used at Step S002. When it is judged that there is something unusual in the medicine ejection unit 2, the user is notified of that somehow and is urged to replace the unit (Step S013).

Similarly to the remaining amount of the medicine, in order to reserve power necessary for at least one inhalation, similarly to Step S003, the remaining power in the battery is checked (Step S011). When it is judged that the battery can afford no more preliminary ejection, display urging an immediate inhalation operation or a replacement of the battery is somehow carried out (Step S014).

When the battery can afford some more preliminary ejection, it is decided whether or not the number of the preliminary ejections described in the above reaches the upper limit expressed as (C−A)/B (Step S012). When the number of the preliminary ejections does not reach the upper limit, monitoring of the switch for the preliminary ejection (Step S016) and monitoring of the inhalation (Step S017) are repeated. When the switch for the preliminary ejection is turned on, the process beginning at Step S007 where display is carried out to notify the user that the preliminary ejection is under way is repeated.

By repeating the preliminary ejection operations, the number of the preliminary ejections reaches the upper limit in the end. In this case, display urging an immediate inhalation operation or a replacement of the medicine ejection unit 2 is carried out (Step S015) and start of inhalation by the user is waited for.

When inhalation is detected, display notifying the user that the ejection is under way (Step S018) and ejection (Step S019) are carried out. Then, the power is turned off (Step S020) and the process ends (Step S021).

When the medicine ejection unit 2 is not of a one-time-use disposable type, the history of the number of the preliminary ejection operations and the number of the inhalations is maintained when the power is turned off (Step S020) or when the process ends (Step S021). In this way, the remaining amount of the liquid medicine is kept track of in preparation for the next use.

Further, after the ejection (Step S019) ends, check of the medicine ejection unit 2 similar to that at Step S002 or S010 and/or check of the remaining power in the battery similar to that at Step S003 or S011 may be carried out.

In order to carry out the operation described in the above, for example, a program for carrying out the above-mentioned procedure may be implemented on a memory of the control means including a CPU. According to this embodiment, since the inhalation operation cannot be carried out until after at least one preliminary ejection operation is carried out, the reliability of the normal ejection is further improved and comfort can be given to the user. In this embodiment, too, the user can visually check the ejection state of the minute liquid droplets with a relatively simple structure in which the medicine ejection unit 2 is controllable so that the preliminary ejection is carried out and the light emitting means 4 is provided so as to check the ejection state.

Further, a method of improving the visibility in the visual check may be devised. For example, by structuring the direction of the visual check and the direction of the emitted light so as not to be opposed to each other, the emitted light does not have to be directly viewed and the visibility can be improved.

### Embodiment 3

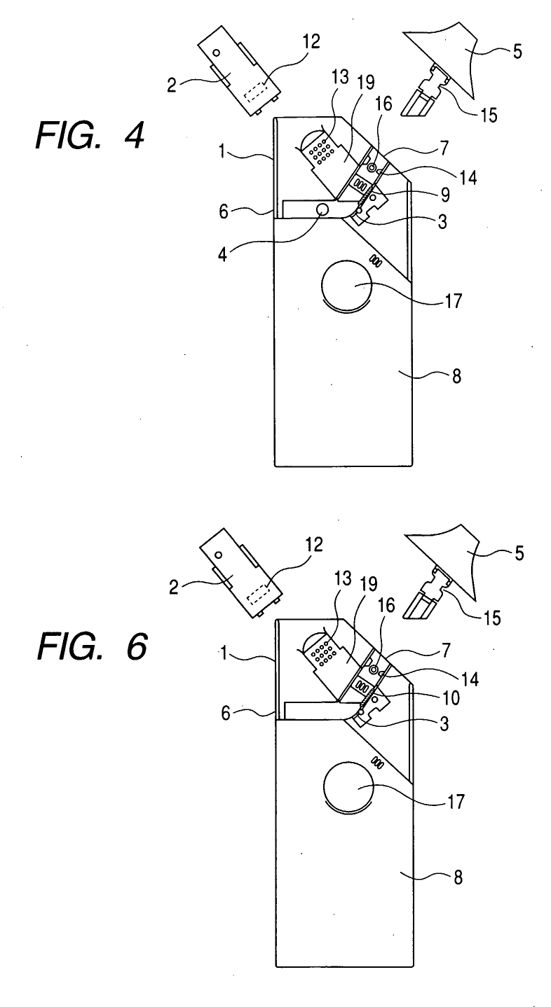

FIGS. 2 and 4 illustrate a structure of a main portion of a third embodiment of the present invention. In FIG. 4, reference numeral 9 denotes a CCD sensor, in particular, a window where the CCD sensor picks up an image.

In this embodiment, normal ejection when the medicine is inhaled is started by detecting means such as the negative pressure sensor 16 in association with the inhalation operation. On the other hand, when preliminary ejection for checking the ejection state of the head portion 12 is carried out, the ejection is started by, for example, operating a switch by which the user can arbitrarily instruct the start. The amount of the preliminary ejection may be set on the side of the device to be equal to or lower than the amount of the normal ejection, or may be adjusted by the user's operation of the switch. In this case, the light emitting means 4 may emit light simultaneously with the start of the preliminary ejection, or the user may make the light emitting means 4 to emit light by operating another switch.

In the preliminary ejection, unlike the case of the normal ejection, air flow is not generated and the ejected medicine drifts like a mist in the air flow path 3 near the head portion 12. The medicine liquid droplets ejected at the time of the preliminary ejection and drifting like a mist in a relatively dark place is illuminated by the light emitting means 4 and the image thereof is picked up by the CCD sensor 9, and the image displayed by display means (not shown) is observed. As a result, the user himself/herself can visually check the state to see whether or not the preliminary ejection is carried out. Here, when it can be confirmed the preliminary ejection is carried out correctly, the operation may proceed to the normal ejection to carry out the inhalation of the liquid medicine. When it is not confirmed that the preliminary ejection is carried out correctly, appropriate measures may be taken such as check of the preliminary ejection once again. When a successful preliminary ejection still cannot be confirmed, the medicine ejection unit 2 may be replaced.

In so far as the image of the medicine liquid droplets ejected at the time of the preliminary ejection can be picked up, the CCD sensor 9 may be disposed anywhere on the passage means 3. Further, light emitted by the light emitting means 4 need not be necessarily visible light in so far as the CCD sensor 9 can pick up the image therewith and the picked up image can be displayed by the display means. In so far as the user can observe the preliminary ejection correctly, the image displayed by the display means may be the image picked up by the CCD sensor 9 as it is, or may be an image after some kind of image processing.

### Embodiment 4

Next, a fourth embodiment of the present invention is described. An outer structure of this embodiment is also as illustrated in FIGS. 2 and 4. In this embodiment, control means is structured such that the preliminary ejection and the normal ejection are carried out unlike those in the third embodiment.

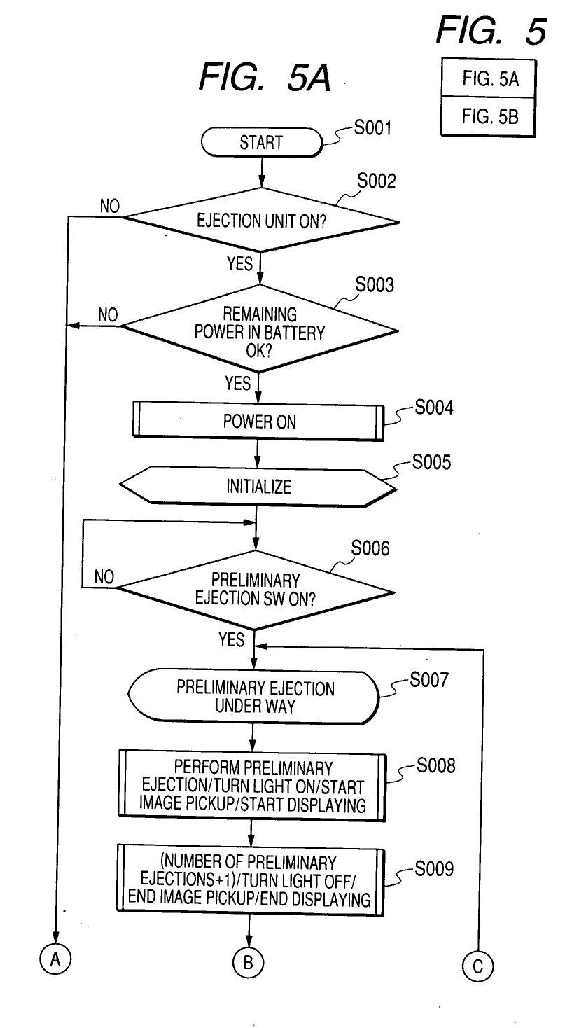

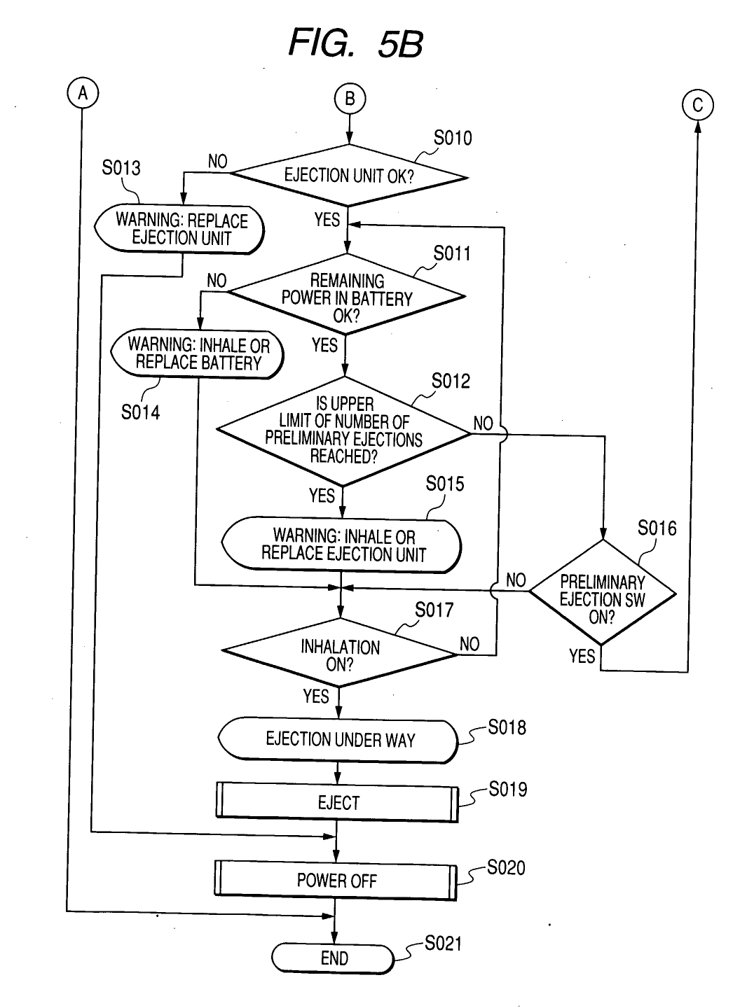

FIGS. 5A and 5B are operation flow charts of a medicine ejection device 1 of this embodiment. First, the device 1 enters a use starting state by operation such as pressing a power on/off switch by a user (Step 001). After the use of the device starts, the device 1 checks whether or not there is a medicine ejection unit 2 (Step S002). If the answer is NO, the process proceeds to an end as it is (Step S021). Detecting means for detecting whether or not there is the medicine ejection unit 2 at an ejection unit attaching portion 19 can be materialized by, for example, when the medicine ejection unit 2 ejects the medicine by a bubble jet system, measuring the resistance of a heater as ejection energy generating means.

When there is the medicine ejection unit 2, the remaining power in the battery is checked (Step S003). If the answer is NO, the process proceeds to the end (Step 021). In this case, display means 18 (see FIG. 2) or the like may be used to provide an indication urging the user to replace or recharge the battery. When it is judged that the remaining power in the battery is enough for at least one preliminary ejection and one inhalation operation, the power is turned on (Step S004) and the device is initialized (Step S005). When the medicine ejection unit 2 is of a one-time-use disposable type, the number of the preliminary ejection operations is reset in the initialization at Step S005.

After the reset at Step S005 is completed, in order to improve the reliability of the ejection, it is necessary to carry out the preliminary ejection operation at least once. Therefore, judgment whether a switch for the preliminary ejection is turned on or not at Step S006 may be automated.

When start of the preliminary ejection is instructed automatically or manually, display is carried out to notify the user that the preliminary ejection is under way (Step S007), and, in order to start the check operation, the preliminary ejection from the ejection head 12 and emission of light by the light emitting means 4 are carried out, the CCD sensor 9 starts to pick up the image, and the image is displayed by display means (not shown) (Step S008). Here, the user visually checks the state of the preliminary ejection through the air flow path 3.

It is assumed that the conditions of the ejection such as drive frequency and drive time suitable for visual observation differ. If the conditions of the ejection change, the amount of the medicine and the electric power consumed in the preliminary ejection also change. In order to reserve an amount of the medicine necessary for at least one inhalation, a controlling function operated by the user to limit the number of the preliminary ejections is necessary. It is desirable that the amount of the medicine consumed in the preliminary ejection is smaller than that necessary for the inhalation. The preliminary ejection is allowed to be carried out up to (C−A)/B (>1) times, wherein A is an amount necessary for the inhalation, B is an amount consumed in the preliminary ejection, and C is the total amount of the medicine contained in the medicine ejection unit 2 or the maximum amount used in one ejection. After the preliminary ejection ends, the number of the preliminary ejection operations is counted, the light emitting means 4 is shut off, and the image pickup by the CCD sensor 9 and the display by the display means (not shown) end to end the check operation (Step S009).

Since there is a fear in that the preliminary ejection causes something unusual in the drug ejection unit 2 to pose a problem in inhaling, checking means for the medicine ejection unit 2 is provided in the medicine ejection device 1 to check the medicine ejection unit 2 after the preliminary ejection ends (Step S010). The checking means itself may be the same as the means for checking whether or not there is the medicine ejection unit 2 used at Step S002. When it is judged that there is something unusual in the medicine ejection unit 2, the user is notified of that somehow and is urged to replace the unit (Step S013).

Then, similarly to the remaining amount of the medicine, in order to reserve power necessary for at least one inhalation, similarly to Step S003, the remaining power in the battery is checked (Step S011). When it is judged that the battery can afford no more preliminary ejection, display urging an immediate inhalation operation or a replacement of the battery is somehow carried out (Step S014).

When the battery can afford some more preliminary ejection, it is decided whether or not the number of the preliminary ejections described in the above reaches the upper limit expressed as (C−A)/B (Step S012). When the number of the preliminary ejections does not reach the upper limit, monitoring of the switch for the preliminary ejection (Step S016) and monitoring of the inhalation (Step S017) are repeated. When the switch for the preliminary ejection is turned on, the process beginning at Step S007 where display is carried out to notify the user that the preliminary ejection is under way is repeated.

By repeating the preliminary ejection operations, the number of the preliminary ejections reaches the upper limit in the end. In this case, display urging an immediate inhalation operation or a replacement of the medicine ejection unit 2 is carried out (Step S015) and start of inhalation by the user is waited for.

When inhalation is detected, display notifying the user that the ejection is under way (Step S018) and ejection (Step S019) are carried out. Then, the power is turned off (Step S020) and the process ends (Step S021).

When the medicine ejection unit 2 is not of a one-time-use disposable type, the history of the number of the preliminary ejection operations and the number of the inhalations is maintained when the power is turned off (Step S020) or when the process ends (Step S021). In this way, the remaining amount of the liquid medicine is kept track of in preparation for the next use.

Further, after the ejection (Step S019) ends, check of the medicine ejection unit 2 similar to that at Step S002 or S010 and/or check of the remaining power in the battery similar to that at Step S003 or S011 may be carried out.

In order to carry out the operation described in the above, for example, a program for carrying out the above-mentioned procedure may be implemented on a memory of the control means including a CPU. According to this embodiment, since the inhalation operation cannot be carried out until after at least one preliminary ejection operation is carried out, the reliability of the normal ejection is further improved and comfort can be given to the user. Further, a method of improving the state of the image pickup may be devised. For example, by structuring the direction of the image pickup and the direction of the emitted light so as not to be opposed to each other, the emitted light is not directly picked up and the visibility can be improved. Further, by using light invisible to the naked eye such as infrared rays, the sensitivity of the image pickup carried out by the CCD sensor 9 can be improved.

### Embodiment 5

FIGS. 2 and 6 illustrate a structure of a main portion of a third embodiment of the present invention. In FIG. 6, reference numeral 10 denotes a microphone. The head portion 12 carries out the ejection by a bubble jet system. In this embodiment, normal ejection when the medicine is inhaled is started by detecting means such as the negative pressure sensor 16 in association with the inhalation operation. On the other hand, when preliminary ejection for checking the ejection state of the head portion 12 is carried out, the ejection is started by, for example, operating a switch by which the user can arbitrarily instruct the start. The amount of the preliminary ejection may be set on the side of the device to be equal to or lower than the amount of the normal ejection, or may be adjusted by the user's operation of the switch.

When the ejection is carried out by a bubble jet system, the medicine foams. When the medicine is defoamed, minute sound (cavitation noise) is generated. The sound level of the cavitation noise varies depending on the number of nozzles which eject the medicine at the same time, and the frequency of the generated sound is predicted by the drive period and the number of the divided blocks.

The microphone 10 collects cavitation noise which is amplified by amplifying means (not shown) to be outputted from a speaker (not shown). By hearing the sound, whether the preliminary ejection is carried out or not can be checked. Here, when it can be confirmed that the preliminary ejection is carried out correctly, the operation may proceed to the normal ejection to carry out the inhalation of the liquid medicine. When it cannot be confirmed that the preliminary ejection is carried out correctly, appropriate measures may be taken such as check of the preliminary ejection once again. When a successful preliminary ejection sill cannot be confirmed, the medicine ejection unit 2 may be replaced.

When the sound collected by the microphone 10 is amplified by the amplifying means, by shifting the whole spectra of the inputted sound to the side of the higher frequency or to the side of the lower frequency by a minute amount, a howl which can be caused between the speaker (not shown) and the microphone 10 can be prevented. Further, the means by which the user hears the outputted sound may be an earphone instead of the speaker.

### Embodiment 6

Next, a sixth embodiment of the present invention is described. An outer structure of this embodiment is also as illustrated in FIGS. 2 and 4. In this embodiment, control means is structured such that the preliminary ejection and the normal ejection are carried out unlike those in the fifth embodiment.

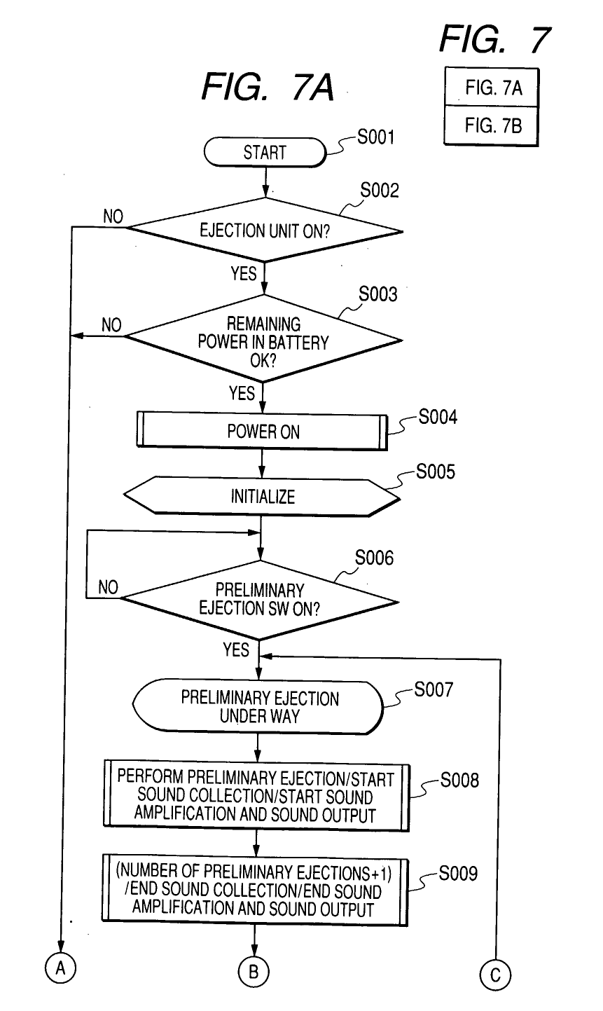

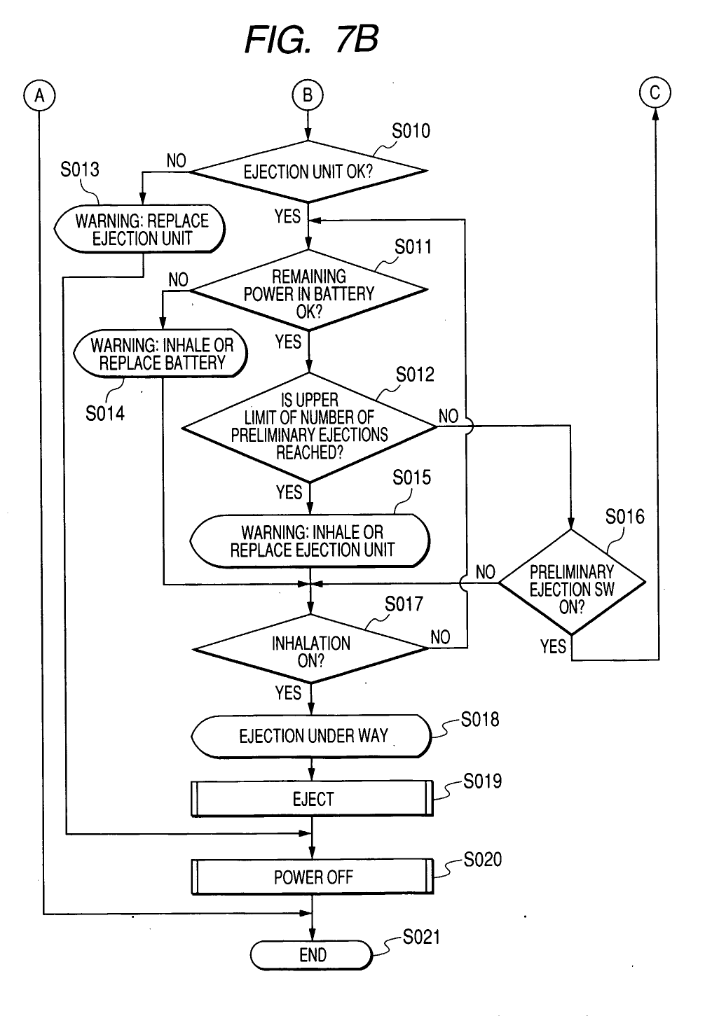

FIGS. 7A and 7B are operation flow charts of a medicine ejection device 1 of this embodiment. First, the device 1 enters a use starting state by an operation such as pressing a power on/off switch by a user (Step 001). After the use of the device starts, the device 1 checks whether or not there is a medicine ejection unit 2 (Step S002). If the answer is NO, the process proceeds to an end as it is (Step S021). Detecting means for detecting whether or not there is the medicine ejection unit 2 at an ejection unit attaching portion 19 can be materialized by, for example, when the medicine ejection unit 2 ejects the medicine by a bubble jet system, measuring the resistance of a heater as ejection energy generating means.

When there is the medicine ejection unit 2, the remaining power in the battery is checked (Step S003). If the answer is NO, the process proceeds to the end (Step 021). In this case, display means 18 (see FIG. 2) or the like may be used to provide an indication urging the user to replace or recharge the battery. When it is judged that the remaining power in the battery is enough for at least one preliminary ejection and one inhalation operation, the power is turned on (Step S004) and the device is initialized (Step S005). When the medicine ejection unit 2 is of a one-time-use disposable type, the number of the preliminary ejection operations is reset in the initialization at Step S005.

After the initialization at Step S005 is completed, in order to improve the reliability of the ejection, it is necessary to carry out the preliminary ejection operation at least once. Therefore, judgement whether a switch for the preliminary ejection is turned on or not at Step S006 may be automated.

When start of the preliminary ejection is instructed automatically or manually, display is carried out to notify the user that the preliminary ejection is under way (Step S007), and, in order to start the check operation, the preliminary ejection from the ejection head 12 and emission of light by the light emitting means 4 are carried out, sound is collected by the microphone 10, and amplification and output of the sound is carried out (Step S008). Here, the user checks the state of the preliminary ejection through a speaker (not shown).

In the preliminary ejection, it is assumed that the conditions of the ejection such as drive frequency and drive time suitable for auditory observation differ depending on the kind of the medicine, the prescription, an additive, and the like. If the conditions of the ejection change, the amount of the medicine and the electric power consumed in the preliminary ejection also change. In order to reserve an amount of the medicine necessary for at least one inhalation, a controlling function operated by the user to limit the number of the preliminary ejections is necessary. It is desirable that the amount of the medicine consumed in the preliminary ejection is smaller than that necessary for the inhalation. The preliminary ejection is allowed to be carried out up to (C−A)/B (>1) times, wherein A is an amount necessary for the inhalation, B is an amount consumed in the preliminary ejection, and C is the total amount of the medicine contained in the medicine ejection unit 2 or the maximum amount used in one ejection. After the preliminary ejection ends, the number of the preliminary ejection operations is counted, and the sound collection, amplification, and output end to end the check operation (Step S009).

Since there is a fear in that the preliminary ejection causes something unusual to pose a problem in inhaling, checking means for the medicine ejection unit 2 is provided in the medicine ejection device 1 to check the medicine ejection unit 2 after the preliminary ejection ends (Step S010). The checking means itself may be the same as the means for checking whether or not there is the medicine ejection unit 2 used at Step S002. When it is judged that there is something unusual in the medicine ejection unit 2, the user is notified of that somehow and is urged to replace the unit (Step S013).

Then, similarly to the remaining amount of the medicine, in order to reserve power necessary for at least one inhalation, similarly to Step S003, the remaining power in the battery is checked (Step S011). When it is judged that the battery can afford no more preliminary ejection, display urging an immediate inhalation operation or a replacement of the battery is somehow carried out (Step S014).

When the battery can afford some more preliminary ejection, it is decided whether or not the number of the preliminary ejections described in the above reaches the upper limit expressed as (C−A)/B (Step S012). When the number of the preliminary ejections does not reach the upper limit, monitoring of the switch for the preliminary ejection (Step S016) and monitoring of the inhalation (Step S017) are repeated. When the switch for the preliminary ejection is turned on, the process beginning at Step S007 where display is carried out to notify the user that the preliminary ejection is under way is repeated.

By repeating the preliminary ejection operations, the number of the preliminary ejections reaches the upper limit in the end. In this case, display urging an immediate inhalation operation or a replacement of the medicine ejection unit 2 is carried out (Step S015) and start of inhalation by the user is waited for.

When inhalation is detected, display notifying the user that the ejection is under way (Step S018) and ejection (Step S019) are carried out. Then, the power is turned off (Step S020) and the process ends (Step S021).

When the medicine ejection unit 2 is not of a one-time-use disposable type, the history of the number of the preliminary ejection operations and the number of the inhalations is maintained when the power is turned off (Step S020) or when the process ends (Step S021). In this way, the remaining amount of the liquid medicine is kept track of in preparation for the next use.

Further, after the ejection (Step S019) ends, check of the medicine ejection unit 2 similar to that at Step S002 or S010 and/or check of the remaining power in the battery similar to that at Step S003 or S011 may be carried out.

In order to carry out the operation described in the above, for example, a program for carrying out the above-mentioned procedure may be implemented on a memory of the control means including a CPU. According to this embodiment, since the inhalation operation cannot be carried out until after at least one preliminary ejection operation is carried out, the reliability of the normal ejection is further improved and comfort can be given to the user. Further, although the collection, amplification, and output of the sound is started at the START step (S008) and is ended at the END step (S009), since all that is required is to start/end the output of the sound, the collection and amplification of the sound are not necessary required to be controlled.

The present invention is not limited to the above embodiments and various changes and modifications can be made within the spirit and scope of the present invention. Therefore, to apprise the public of the scope of the present invention, the following claims are made.

This application claims priority from Japanese Patent Application No. 2005-096590 filed Mar. 30, 2005, which is hereby incorporated by reference herein.

1-10. (canceled)

11. A liquid medicine ejection device for ejecting a liquid medicine as liquid droplets to be inhaled by a user, comprising:
a passage for enabling a preliminary ejection to be visually checked from the outside when the preliminary ejection is carried out, the preliminary ejection ejecting an amount of the liquid medicine smaller than an amount ordinarily ejected.

12. The liquid medicine ejection device according to claim 11, further comprising light emitting means for illuminating around an ejection portion with visible light.

13. The liquid medicine ejection device according to claim 12, wherein said passage serves also as a flow path of airflow containing liquid droplets when the liquid droplets are inhaled.

14. A liquid medicine ejection device for ejecting a liquid medicine as liquid droplets to be inhaled by a user, comprising:
means for taking an image in the vicinity of an ejection portion; and
means for displaying the picked up image,
wherein a preliminary ejection can be checked, the preliminary ejection ejecting an amount of the liquid medicine smaller than an amount ordinarily ejected.

15. The liquid medicine ejection device according to claim 14, further comprising light emitting means for illuminating around the ejection portion.

16. A liquid medicine ejection device for ejecting a liquid medicine as liquid droplets to be inhaled by a user, comprising:
means for collecting sound generated upon ejection; and
means for outputting the collected sound to the user,
wherein a preliminary ejection can be checked, the preliminary ejection ejecting an amount of the liquid medicine smaller than an amount ordinarily ejected.

17. The liquid medicine ejection device according to claim 16, further comprising means for amplifying sound collected by said means for collecting sound, wherein sound amplified by said means for amplifying sound is outputted by said means for outputting the collected sound.
